(19) United States
(12) Patent Application Publication
(10) Pub. No.: US 2025/0281735 A1
(43) Pub. Date: Sep. 11, 2025

(54) Cloud-based AI intelligent electrical stimulation system and control method

(71) Applicant: Fasikl Incorporated, DALLAS, TX (US)

(72) Inventor: Zhi Yang, DALLAS, TX (US)

(21) Appl. No.: 19/215,814

(22) Filed: May 22, 2025

(30) Foreign Application Priority Data
Apr. 2, 2025 (CN) 202510406554.X

(51) Int. Cl. A61N 1/04; A61N 1/36; A61N 1/37

(52) U.S. Cl. CPC A61N 1/0484; A61N 1/36135; A61N 1/3702

(57) ABSTRACT

Disclosed are a cloud-based AI intelligent electrical stimulation system and control method. The cloud-based AI intelligent electrical stimulation system includes: a wrist stimulator, including a first acquisition module and a stimulation module; a mobile terminal in communication connection with the wrist stimulator; a cloud server in communication connection with the mobile terminal, where the AI model module outputs a stimulation parameter set based on the first data and pre-stored user data, and the first data is transmitted by the wrist stimulator to the cloud server through the mobile terminal; and the optimization processing module adjusts the stimulation parameter set according to user tolerance data and transmits the adjusted stimulation parameter set to the wrist stimulator. The cloud-based AI intelligent electrical stimulation system enables precise monitoring of user tremors, personalized electrical stimulation treatment, and real-time output of corresponding stimulation parameter sets, and personalized and optimized therapies are available.

### TECHNICAL FIELD

The present disclosure relates to the technical field of neurostimulation devices, and in particular to a cloud-based AI intelligent electrical stimulation system and control method.

### BACKGROUND

Essential tremor (ET) and Parkinson's disease (PD) are two common and significantly impactful neurological diseases. ET is a prevalent movement disorder with a global prevalence rate of 0.4%-5%, the prevalence rate increases with age, and there are about 10 million ET patients only in the United States. Clinical manifestations of ET primarily include postural or kinetic tremors of hands and arms, which usually affect lower limbs, heads, oral and facial areas, and voices. Such symptoms tend to worsen in case of attention focusing, mental stress, fatigue or hunger, which severely affect the life quality of a patient, and potentially lead to anxiety, depression, sleep disorders, social withdrawal, and lack of confidence. Therapeutic drugs of ET include β-blockers (propranolol), primidone, and botulinum toxin injections, but they exhibit ineffective or suboptimal efficacy for 30-50% of patients. Surgical treatments include thalamic radiofrequency ablation and deep brain stimulation, but various risks exist.

As of 2021, approximately 6-10 million people from many countries were diagnosed with PD as the second most prevalent neurodegenerative disease globally. The symptoms of PD are diverse, including tremors, rigidity, bradykinesia, postural instability, psychiatric disorders and autonomic dysfunction. PD patients often suffer from limb rigidity, sleep disorders, cognitive impairments, anxiety, depression and the like. Therapeutic drugs of PD such as Madopar, dopamine receptor agonists and the like relieve symptoms of 80-97% of PD patients by 60-80%, but cause side effects such as arrhythmias and bradycardia; and surgical treatments (such as neurodestructive surgeries and deep brain stimulation) also carry potential risks of hemorrhage and infection.

Tremors cannot be effectively suppressed, thereby necessitating the urgent development of effective, low-side-effect, non-invasive therapies. The application of a peripheral nerve electrical stimulation therapy dates back to the 1990s or earlier. However, many problems exist in the field of peripheral nerve electrical stimulation, including: non-adaptive parameter settings and significant variations in therapeutic responses of different users; lack of long-term clinical data on efficacies and potential risks; local skin irritation, pain, or numbness caused by electrical stimulation; diminished therapeutic efficacy to some users due to drug tolerance; diminished therapeutic efficacy or side effects caused by complex parameter optimization; and poor user experience from suboptimal wearability and operational complexity.

In view of this, it is urgently needed to develop an improved peripheral nerve electrical stimulation therapy to overcome the above defects and better serve users.

### SUMMARY

In view of the defects in the prior art, a first objective of the present disclosure is to provide a cloud-based AI intelligent electrical stimulation system capable of dynamically matching a stimulation parameter set of electrical stimulation pulses according to drug tolerance of a user and enhancing user experience.

A second objective of the present disclosure is to provide a cloud-based AI intelligent electrical stimulation control method.

In order to achieve the above objectives, the present disclosure adopts the following technical solutions:

In an aspect, the present disclosure provides a cloud-based AI intelligent electrical stimulation system, including:
a wrist stimulator, including a first acquisition module and a stimulation module, where the first acquisition module is configured to collect first data, and the stimulation module is configured to apply electrical stimulation pulses to one or more target nerves; the first data includes one or more of tremor data and physiological data;
a mobile terminal in communication connection with the wrist stimulator;
] a cloud server in communication connection with the mobile terminal, including an AI model module and an optimization processing module;
the AI model module outputs a stimulation parameter set based on the first data and pre-stored user data, and the first data is transmitted by the wrist stimulator to the cloud server through the mobile terminal; the stimulation parameter set includes one or more control parameters of the electrical stimulation pulses; and
the optimization processing module adjusts the stimulation parameter set according to user tolerance data and transmits the adjusted stimulation parameter set to the wrist stimulator, such that the wrist stimulator outputs electrical stimulation pulses based on the stimulation parameter set.

Preferably, the mobile terminal further generates pre-stored user data and/or user tolerance data based on consultation results and transmits the data to the cloud server.

Preferably, after receiving the stimulation parameter set sent by the cloud server, the mobile terminal converts the stimulation parameter set into a waveform graph for display, and marks a corresponding user tolerance benchmark identifier and an original optimal stimulation identifier.

Preferably, the mobile terminal is also configured to receive the user's daily tolerance data and transmit the data to the cloud server, where the optimization processing module further adjusts the stimulation parameter set based on the daily tolerance data.

Preferably, a wearable accessory is further included, and the wearable accessory is provided with a second acquisition module in communication connection with the mobile terminal or the wrist stimulator for detecting second data of the user; and the second data includes one or more of tremor data and physiological data.

When the wearable accessory is in communication connection with the mobile terminal, the mobile terminal, upon receiving the second data, determines the user's physical condition according to the second data and transmits the second data to the cloud server, where the AI model module outputs a stimulation parameter set based on the first data, the second data and the pre-stored user data; and
when the wearable accessory is in communication connection with the wrist stimulator, the wrist stimulator transmits both the second data received and the first data collected to the cloud server through the mobile terminal, where the AI model module outputs a stimulation parameter set according to the first data, the second data, and the pre-stored user data.

Preferably, the wearable accessory includes one or more of a bracelet, glasses, an earphone, a ring, a headband, a waistbelt, a patch, and jewelry.

Preferably, the target nerve is one of a radial nerve, a median nerve, or an ulnar nerve.

Preferably, a training method for the AI model module includes:
collecting multimodal data to construct a training set, where the multimodal data includes data of the user's age, medical history, tremor type, real-time movement status, electrical stimulation response, and electrical stimulation parameter set;
preprocessing the training set to balance label distribution; and
training an initialized neural network based on the training set to obtain the AI model module.

Preferably, the AI model module includes:
a transfer learning unit, configured to adapt an initial stimulation strategy for a new user based on a cross-user data generalization model; and
a reinforcement learning unit, configured to optimize a long-term efficacy indicator through a reward function according to operational detection data collected by the wrist stimulator each time and a corresponding stimulation parameter set and configure a personalized stimulation strategy.

Preferably, a waveform of the electrical stimulation pulse is one or a combination of more of a biphasic square wave, a sine wave, a pulse wave, a triangular wave, and a sharp wave, with a frequency range of 1-1000 Hz, and a current intensity of 0.1-20 mA.

Preferably, the first data further includes physiological data; and
the physiological data includes one or more of skin impedance data, therapeutic response data, electromyographic signal data, neural signal data, skin temperature data, and blood oxygen saturation data.

Preferably, the stimulation module includes a plurality of electrode wristbands tailored to different wrist sizes, and each of the electrode wristbands is provided with one or more electrode sets for applying electrical stimulation to the target nerve; and the electrode set comprises one or more electrode pads, and one electrode pad corresponds to one target nerve.

In a further aspect, the present disclosure provides a cloud-based AI intelligent electrical stimulation control method, and the method includes:
collecting first data of the user through the wrist stimulator, where the first data includes one or more of tremor data and physiological data;
uploading the first data to the cloud server through the mobile terminal to generate a stimulation parameter set through the AI model module, where the first data is transmitted by the wrist stimulator to the cloud server through the mobile terminal, and the stimulation parameter set includes one or more control parameters of the electrical stimulation pulses; and
adjusting the stimulation parameter set through the optimization processing module according to user tolerance data, and transmitting the adjusted stimulation parameter set to the wrist stimulator, such that the wrist stimulator outputs electrical stimulation pulses based on the stimulation parameter set.

Compared with the prior art, the cloud-based AI intelligent electrical stimulation system and control method provided by the present disclosure have the following beneficial effects:
1. Precise personalized treatment: the AI model module generates a customized stimulation plan by analyzing the user's historical data and real-time feedback, thereby avoiding the traditional “one-size-fits-all” approach and enhancing stimulation effectiveness.
2. The cloud-based AI intelligent electrical stimulation system enables precise monitoring of user tremors, personalized electrical stimulation treatment, and real-time output of corresponding stimulation parameter sets according to user tolerance, and personalized and optimized therapies provide more effective treatment options for users with ET and PD.

### BRIEF DESCRIPTION OF THE DRAWINGS

FIG. 1 is a structural block diagram of a cloud-based AI intelligent electrical stimulation system provided by the present disclosure.

FIG. 2 is an operational flowchart for setting user tolerance data provided by the present disclosure.

FIGS. 3a-3b illustrate functional interfaces of a mobile terminal for adjusting user tolerance data provided by the present disclosure.

FIGS. 4a-4b illustrate display interfaces of a mobile terminal for stimulation provided by the present disclosure.

FIG. 5 is a flowchart of a cloud-based AI intelligent electrical stimulation control method provided by the present disclosure.

### DETAILED DESCRIPTIONS OF THE EMBODIMENTS

To make objectives, technical solutions and effects of the present disclosure clearer and more explicit, the present disclosure will be further described in detail with reference to accompanying drawings and specific examples. It should be understood that specific examples described herein are merely used to explain the present disclosure, and are not intended to limit the present disclosure.

Those skilled in the art should understand that the general description above and the detailed description below are exemplary and illustrative specific examples of the present disclosure and are not intended to limit the present disclosure.

Herein, the terms “including”, “comprising” or any other variants thereof are intended to cover non-exclusive inclusion, such that a process or method that includes a list of steps not only includes those steps, and but also may include other steps that are not explicitly listed or inherent to such process or method. Similarly, in the entire specification, phrases such as “in one example” or “in another example” and similar words may, but not necessarily, refer to the same example.

Unless otherwise defined, all technical and scientific terms used herein have the same meanings usually understood by those ordinarily skilled in the art to which the present disclosure belongs.

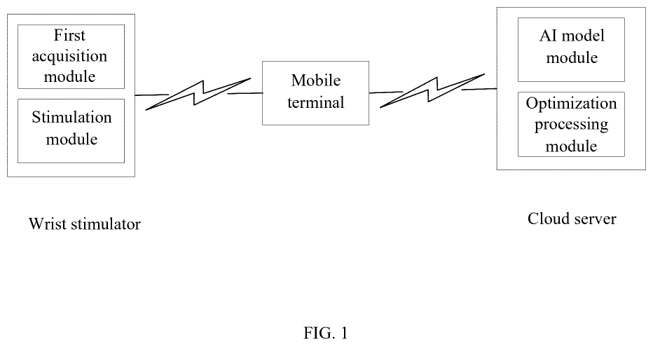

With reference to FIG. 1, the present disclosure provides a cloud-based AI intelligent electrical stimulation system, including:
a wrist stimulator, including a first acquisition module and a stimulation module, where the first acquisition module is configured to collect first data, and the stimulation module is configured to apply electrical stimulation pulses to one or more target nerves; the first data includes one or more of tremor data and physiological data;
a mobile terminal in communication connection with the wrist stimulator;
a cloud server in communication connection with the mobile terminal, including an AI model module and an optimization processing module;
the AI model module outputs a stimulation parameter set based on the first data and pre-stored user data, and the first data is transmitted by the wrist stimulator to the cloud server through the mobile terminal; the stimulation parameter set includes one or more control parameters of the electrical stimulation pulses; and
the optimization processing module adjusts the stimulation parameter set according to user tolerance data and transmits the adjusted stimulation parameter set to the wrist stimulator, such that the wrist stimulator outputs electrical stimulation pulses based on the stimulation parameter set.

Preferably, the user tolerance data includes one or more parameters from the stimulation parameter set. For example, when parameters of the stimulation parameter set include stimulation current, a pulse amplitude, a frequency, an interval, a waveform shape, a stimulation pulse count, a stimulation channel sequence and the like, the user data includes one or more of the stimulation current, the pulse amplitude, the frequency, the interval, the waveform shape, the stimulation pulse count, and the stimulation channel sequence.

In some examples, when adjusting the stimulation parameter set according to user tolerance data, all parts of the stimulation parameter set that exceed the user tolerance data are reduced to the user tolerance data or made below the user tolerance data. For example, the user tolerance data includes the stimulation current with a specific value of 3.5 mA, and when a value of partial stimulation current indicated in the stimulation parameter set is 4 mA, the value of stimulation current will be lowered to 3.5 mA or below 3.5 mA (of course, the value of stimulation current cannot be too low, and is generally not much different from that in the user tolerance data, e.g., 3.4 mA). When the user tolerance data includes other parameters, the frequency is fine-tuned from original 50 Hz to 48 Hz. That is to say, a stimulation parameter set output by the cloud server is an optimal stimulation scheme suitable for a current user.

In some examples, the AI model generally generates a stimulation parameter set at a predetermined interval (e.g., every 1 s to 10 min) and outputs same to the optimization processing module.

In some examples, during operation of the wrist stimulator, a backup waveform is generated at a regular interval, and when the AI model module determines that a difference between the backup waveform and the current stimulation parameter set is larger than a preset threshold, the backup waveform is output to the optimization processing module to replace the current stimulation parameter set with a new one.

Through the above specific embodiment, the cloud-based AI intelligent electrical stimulation system enables precise monitoring of user tremors, personalized electrical stimulation treatment, and real-time output of corresponding stimulation parameter sets according to user tolerance, and personalized and optimized therapies provide more effective treatment options for users with essential tremor (ET) and Parkinson's disease (PD).

Preferably, in some examples, the first acquisition module in the wrist stimulator is provided with a high-precision accelerometer and gyroscope, to accurately detect an amplitude, frequency, direction and the like of hand tremors in three-dimensional space. For example, the accelerometer and the gyroscope are integrated at a position of the wrist stimulator close to a wrist joint to capture subtle hand movements to the maximum extent. The accelerometer is configured to perceive acceleration variations in hand movement, and the gyroscope is configured to measure changes in a rotational angle of hand. The accelerometer and the gyroscope cooperate with each other to comprehensively and accurately obtain tremor data.

In some examples, the first acquisition module is provided with a bioelectric sensor, such as an electrocardiogram sensor, a galvanic skin response sensor, and a blood oxygen sensor. The electrocardiogram sensor collects the user's electrocardiogram data through skin contact to understand the user's heart activity status, and detects certain physiological states (such as abnormal heart rates) related to occurrence or exacerbation of tremors, thereby facilitating optimized stimulation in these scenarios. The galvanic skin response sensor is configured to detect changes in skin surface conductivity and reflect the user's emotional stress level, which enhances precise control over the user's real-time status in comprehensive consideration of emotional factors affecting tremor symptoms. Data collection frequencies of these sensors can be adjusted according to actual needs, and are generally set to 1-1000 Hz, to ensure timely and accurate acquisition of physiological data.

In some examples, the stimulation module includes a plurality of electrodes, and the electrodes are made from a flexible material to better fit wrist skin. A layout of the electrodes is carefully designed to ensure that electrical stimulation pulses can be accurately applied to one or more target nerves. For example, for treatment of hand tremors, the electrodes can be distributed at specific locations of the wrist associated with hand nerves, such as trajectory regions of a median nerve and an ulnar nerve. The electrodes are also optimized in size and shape to better fit the user's wrist and ensure effective stimulation of the nerves during electrical stimulation, without excessive pressure on the skin.

Further, the stimulation module outputs electrical stimulation pulses to the target nerves through the electrodes according to the stimulation parameter set received. Parameters of the electrical stimulation pulses, such as the current, frequency and the like, can be precisely adjusted based on the stimulation parameter set. For example, the stimulation parameter set may require outputting electrical stimulation pulses with a frequency of 1-1000 Hz and a current value of 0.1-20 mA, and the stimulation module is capable of accurately outputting corresponding electrical stimulation according to parameter requirements to achieve a desired therapeutic effect.

In some examples, the AI model module of the cloud server receives the first data and the pre-stored user data from the wrist stimulator. The pre-stored user data includes the user's basic information (such as age, gender, medical history, and the like), previous treatment records, and responses to electrical stimulation. The AI model module integrates and preprocesses these data, for example, tremor data is filtered to eliminate noise interference and physiological data is normalized to facilitate subsequent analysis and model computation.

In some examples, the optimization processing module stores user tolerance data, and the user tolerance data may be obtained based on feedback from the user during use or monitoring of the user's physiological responses through the wrist stimulator. For example, when the user feels that an electrical stimulation intensity is too high or too low, the user can provide feedback through a feedback button on the wrist stimulator, or when the wrist stimulator detects abnormal physiological responses such as changes in skin resistance of the user, the user's tolerance to electrical stimulation can be determined.

The optimization processing module adjusts the stimulation parameter set output by the AI model module according to the user tolerance data. When the user's tolerance level is high, an intensity or frequency of electrical stimulation can be appropriately increased; and when the user's tolerance level is low, values of stimulation parameters are correspondingly lowered. The adjusted stimulation parameter set is transmitted to the wrist stimulator via a GPRS network or a Wi-Fi network, such that the wrist stimulator outputs electrical stimulation pulses according to an optimized stimulation parameter set, thereby enhancing the therapeutic effect and user comfort. For example, when the user tolerance data indicates that the user is well-adapted to a current electrical stimulation intensity, the optimization processing module may appropriately adjust the current parameter indicated in the stimulation parameter set and then transmit the adjusted stimulation parameter set to the wrist stimulator.

In some examples, a mobile terminal is further included, and communication connection between the cloud server and the wrist stimulator is implemented through the mobile terminal. Preferably, the mobile terminal communicates with the wrist stimulator through a short-range communication mode, and the short-range communication mode includes Bluetooth communication, 2.4G communication, ZigBee communication, and the like. The mobile terminal communicates with the cloud server via a Wi-Fi or mobile network. The mobile terminal includes portable devices such as a smartphone and a tablet computer, having user-friendly operating interfaces. With the mobile terminal as a communication bridge, the user does not need to learn complex device connections and operation procedures, but can easily control an interaction between the wrist stimulator and the cloud server through a commonly used mobile application. For example, during outdoor activities, the user only needs to activate a corresponding application installed in his/her smartphone to check an operational status of the wrist stimulator and receive updated treatment recommendations from the cloud server, without need to carry any specialized connection device, thereby greatly enhancing use convenience.

In some examples, the mobile terminal further generates pre-stored user data and/or user tolerance data based on consultation results and transmits the data to the cloud server.

In some examples, pre-stored user data transmitted by the mobile terminal is obtained through input by the user.

In some examples, the mobile terminal interfaces with a consultation system of a medical institution. After the user participates in consultation, the consultation system sends consultation results in a predetermined data format to the mobile terminal. For example, the consultation system, through a secure data interface, transmits a consultation report containing details of the user including diagnosis results, treatment recommendations and an estimated treatment cycle to a designated application on the mobile terminal. The application on the mobile terminal parses and sorts out received consultation results. Key symptom descriptions and disease severity grading are extracted for diagnosis results of the user; and medication therapies, surgical intervention feasibility and related precautions are recorded in detail for treatment recommendations. For example, when the consultation results indicate moderate ET of the user, the medication therapy with propranolol at a dose of 20 mg three times per day is recommended. The mobile terminal integrates these information to generate pre-stored user data. The diagnosis results, treatment recommendations and the like correspond to different data fields, which facilitate subsequent identification and use within the system.

Additionally, in some examples, a dedicated user tolerance feedback interface is configured for the mobile terminal's application according to spontaneous user feedback on tolerance data. When receiving the electrical stimulation treatment with the wrist stimulator, the user can provide feedback based on his/her experience at any time through the mobile terminal. For example, option buttons labeled with the interface may present options such as “too high electrical stimulation intensity”, “appropriate electrical stimulation intensity”, “too low electrical stimulation intensity” and “localized discomfort (e.g., skin tingling, numbness, and the like)” are arranged on the interface. The user clicks a corresponding button according to his/her actual experience, and the mobile terminal instantly records feedback from the user.

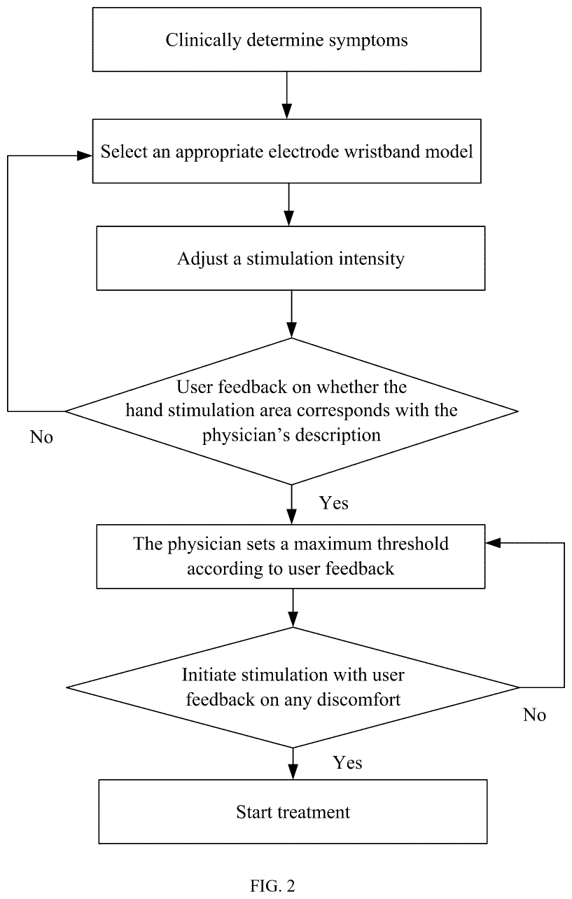

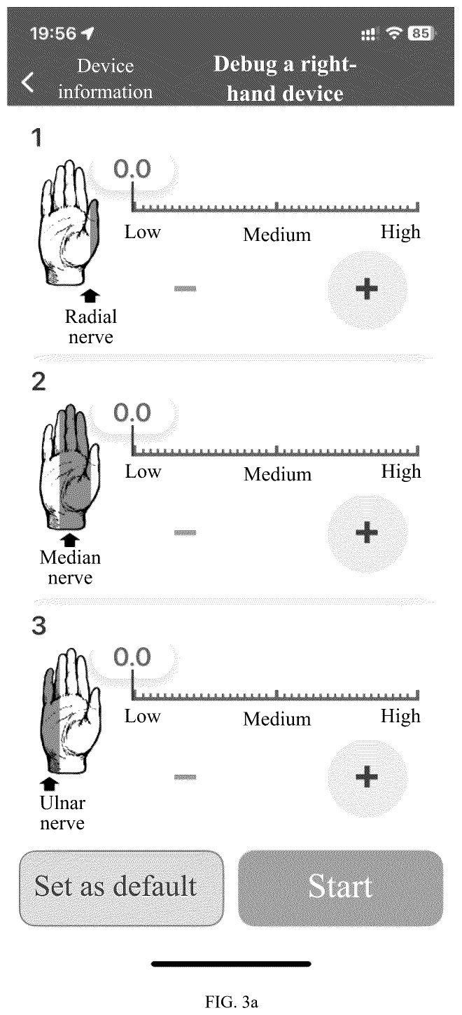

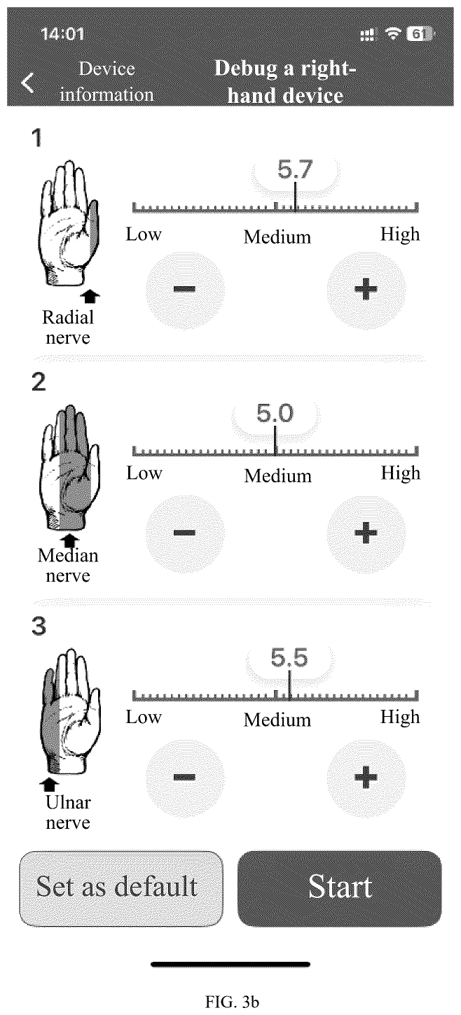

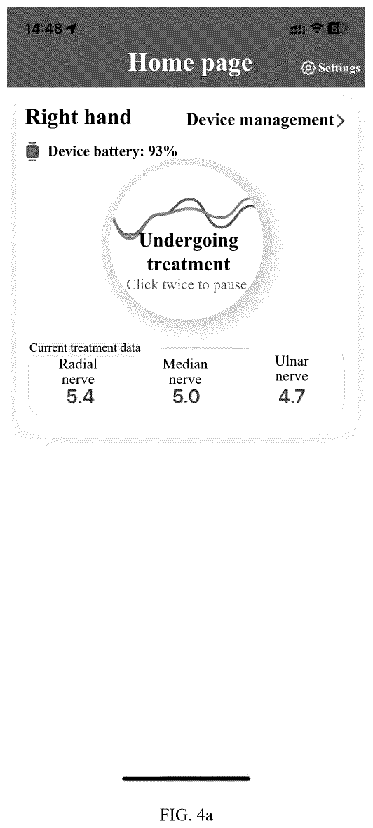

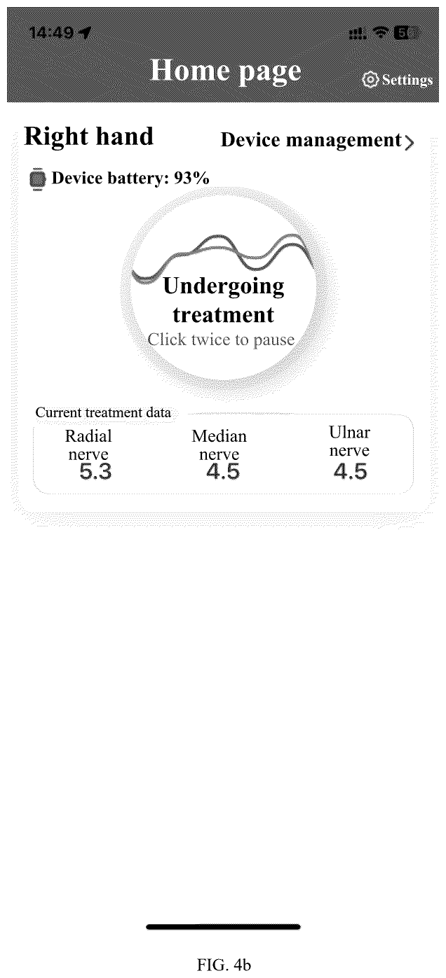

With reference to FIGS. 2, 3a-3b, and 4a-4b, in some examples, user tolerance data is obtained by a physician through clinical inquiries, input through the mobile terminal, and then transmitted to the cloud server. Specifically, the physician determines whether tremors of the user art caused by essential tremor (ET) or Parkinson's disease (PD) according to clinical judgment. The physician measures positions of radial, median, and ulnar nerves at the wrist of the user, as well as a wrist diameter, selects an appropriate electrode model by using an auxiliary tool, and guides the user to properly wear the wrist stimulator. The user then repeats the process until fully mastering skills of proper wearing. During an initial stimulation intensity adaptation on the mobile application, the physician gradually increases the stimulation intensity from low to high for the radial, median, and ulnar nerves. During this process, the physician asks whether a stimulation area reaches a palmar shadow region displayed on the smartphone. When the stimulation area fully covers the palmar shadow region, the physician inquires whether the user feels any discomfort. When discomfort is reported, the physician will reduce the stimulation intensity. Each stimulation adaptation adjustment is performed individually to enhance the user's perceptual awareness of the stimulation area in this process. For example, when calibrating the radial nerve, the median and ulnar nerves are deactivated. When the stimulation area cannot reach the palmar shadow region indicated in FIGS. 3a-3b even at a maximum stimulation intensity, it indicates that a current electrode is not suitable for the user. In this case, the physician will select a different electrode model and repeat the above adaptation steps. When the stimulation area can reach the illustrated palmar shadow region during intensity adjustment, it indicates that the electrode is suitable. The physician will then set a maximum safe threshold for the radial, median, and ulnar nerves respectively according to the user's condition, as shown in FIG. 4b. The maximum safe threshold is an intensity that the user can tolerate. Each time when the physician sets a threshold, the physician will ask the user whether it is suitable or whether further adjustments are needed until the user feels comfortable. When the user feels comfortable, the maximum safe threshold is set as the user tolerance data and transmitted to the cloud server. As compared in FIGS. 4a and 4b, treatment parameters are dynamically adjusted based on a cloud AI model in real time, and these parameters are below the maximum safe threshold, thereby ensuring safety and comfort.

In some examples, during the treatment process, operation of the wrist stimulator can be paused or initiated at any time through the application on the mobile terminal.

The mobile terminal establishes a connection with the cloud server, and the application encapsulates generated pre-stored user data or user tolerance data according to a data transmission protocol specified by the cloud server. For example, the pre-stored user data is packaged into a data packet containing a data header (a data type, a user ID, and the like labeled) and a data body (containing specific consultation result information), and is then sent to the cloud server through a network channel. Upon receiving the data packet, the cloud server unpacks and verifies same to ensure data accuracy and integrity, and stores the pre-stored user data in a corresponding database for later use by the AI model module and the optimization processing module.

In some examples, after receiving the stimulation parameter set sent by the cloud server, the mobile terminal converts the stimulation parameter set into a waveform graph for display, and marks a corresponding user tolerance benchmark identifier and an original optimal stimulation identifier.

Stimulation parameter set reception: The mobile terminal continuously monitors messages from the cloud server via a stable wireless communication link, such as a Wi-Fi or 4G network. When the optimization processing module of the cloud server completes adjustments according to the stimulation parameter set output by the AI model module and the user tolerance data, new stimulation parameter set data is sent to the mobile terminal. For example, at 3:00 PM, the cloud server pushes a set of optimized stimulation parameter set data to the user's mobile terminal, and the data contains detailed parameter information of frequency, current and the like.

Waveform graph conversion and display: A built-in dedicated waveform drawing algorithm is in the application on the mobile terminal. Upon receiving the stimulation parameter set data, the algorithm converts abstract stimulation parameter set data into an intuitive waveform graph according to time series and various parameters in the data. For example, taking time as a horizontal axis and current as a vertical axis, current variation of the stimulation parameter set over time is clearly plotted. In a waveform graph display area, the application presents the waveform in eye-catching colors and line styles to facilitate access by the user.

Adding a user tolerance benchmark identifier and an original optimal stimulation identifier: The mobile terminal retrieves the user's historical tolerance data and original optimal stimulation parameter set data generated by the AI model module from local storage or the cloud server. Based on these data, corresponding identifiers are added to the waveform graph. For example, a red dashed line represents the user tolerance benchmark, e.g., a boundary of stimulation intensity at which the user can continuously tolerate with favorable therapeutic effects achieved during past treatments. A blue solid line represents the original optimal stimulation identifier, and indicates an ideal stimulation parameter set initially provided by the AI model. Through the intuitive identifiers, the user can clearly compare the current stimulation parameter set with the tolerance benchmark for the user and the original optimal stimulation, and have a better understanding of adjustments made to the therapy.

In some examples, the mobile terminal is also configured to receive the user's daily tolerance data and transmit the data to the cloud server, where the optimization processing module further adjusts the stimulation parameter set based on the daily tolerance data.

Receiving daily tolerance data: A designated input area for daily tolerance data is arranged on an application interface on the mobile terminal. When the user experiences changes in the stimulation intensity, comfort and the like during use of the wrist stimulator for the electrical stimulation treatment, the user can input relevant information at any time in the input area. For example, when the user feels that the stimulation intensity is too high during use of the wrist stimulator at 7:00 PM, the user can input daily tolerance data of “too high stimulation intensity at 7:00 PM, accompanied by slight skin tingling” on the mobile terminal. The mobile terminal instantly captures information input by the user and performs preliminary sorting and formatting of the data.

Data transmission to the cloud server: The mobile terminal sends the collated daily tolerance data to the cloud server via a secure network connection. Like other data transmissions, the data is encrypted before sending to ensure data security. For example, the mobile terminal encapsulates a data packet containing a user ID, a timestamp, and detailed descriptions of daily user tolerance according to a data transmission protocol specified by the cloud server, and then sends same through a network channel. Upon receiving the data packet, the cloud server unpacks and verifies same to ensure data accuracy and integrity

Stimulation parameter set adjustment: After the optimization processing module of the cloud server receives the daily tolerance data sent by the mobile terminal, data analysis is performed immediately. According to the user's historical tolerance data and the current treatment stage, the optimization processing module adjusts the stimulation parameter set output by the AI model module by using a specific algorithm. For example, when the user reports that the electrical stimulation intensity is too high, the optimization processing module may appropriately lower a current parameter of the stimulation parameter set, and fine-tune the frequency to meet the user's tolerance needs without prejudice to the therapeutic effect. The adjusted stimulation parameter set is then sent back to the mobile terminal through the cloud server, which realizes real-time optimization of the therapy and enhances both treatment precision and user comfort.

In some examples, a wearable accessory is further included, and the wearable accessory is provided with a second acquisition module in communication connection with the mobile terminal or the wrist stimulator for detecting second data of the user; and the second data includes one or more of tremor data and physiological data.

When the wearable accessory is in communication connection with the mobile terminal, the mobile terminal, upon receiving the second data, determines the user's physical condition according to the second data; the second data is transmitted to the cloud server, and the AI model module outputs a stimulation parameter set based on the first data, the second data and the pre-stored user data; and when the wearable accessory is connected to the mobile terminal, the application on the mobile terminal receives and integrates the data for preliminary analysis. For example, the application compares and analyzes tremor data collected by a smart bracelet with tremor data collected by the wrist stimulator to assess differences in tremor conditions in various body parts of the user. Additionally, physiological data is combined with the user's daily activities, e.g., heart rate, step count and other parameters are used to evaluate whether the user's current physical condition allows continued stimulation. Of course, the process of determining the user's physical condition can also be implemented through the AI model, and a specific process is consistent with previously described steps, with details not described herein again.

When the wearable accessory is in communication connection with the wrist stimulator, the wrist stimulator transmits both the second data received and the first data collected to the cloud server through the mobile terminal, where the AI model module outputs a stimulation parameter set according to the first data, the second data, and the pre-stored user data. By utilizing these more comprehensive data, the AI model of the cloud server can analyze the user's medical condition and therapeutic effect more accurately. For example, by combining heart rate variations detected by the smart bracelet with tremor data from the wrist stimulator, the AI model can determine patterns of tremor occurrence under different physiological states, which provides a more reliable basis for adjusting the electrical stimulation therapy and achieving more personalized and precise treatment.

In some examples, the wearable accessory includes one or more of a bracelet, glasses, an earphone, a ring, a headband, a waistbelt, a patch, and jewelry.

Specifically, the wearable accessory is configured to detect data of the wrist and/or other body parts and has short-range communication functions, such as Bluetooth communication, and can be in communication connection with the mobile terminal (such as a smartphone) or the wrist stimulator, or in direct communication connection with the mobile terminal (the wrist stimulator in this mode is more power-efficient), or in communication connection with the wrist stimulator (power consumption of the wrist stimulator in this mode is higher).

Preferably, the second acquisition module is internally provided with one or more of a high-precision accelerometer, a gyroscope, an electrocardiogram sensor, a galvanic skin response sensor, and a blood oxygen sensor.

In some examples, the physiological data includes one or more of skin impedance data, therapeutic response data, electromyographic signal data, skin temperature data, and blood oxygen saturation data.

In some examples, the target nerve is one of a radial nerve, a median nerve, or an ulnar nerve.

In some examples, a training method for the AI model module includes:
collect multimodal data to construct a training set, where the multimodal data includes data of the user's age, medical history, tremor type, real-time movement status, electrical stimulation response, and electrical stimulation parameter set;
preprocess the training set to balance label distribution; and
train an initialized neural network based on the training set to obtain the AI model module. Preferably, the neural network includes a deep learning network, a naive Bayesian network and the like.

In some examples, the AI model module includes:
a transfer learning unit, configured to adapt an initial stimulation strategy for a new user based on a cross-user data generalization model; and the transfer learning unit first extracts common features from a generalization model of massive cross-user data, and the data includes multimodal information of many users including data of age, medical history, tremor type, and the like. For example, a treatment therapy effective for user groups similar to a new user in age and medical condition can be found in a generalization model constructed based on data of 1000 previous cases. Based on these similar features, an initial stimulation strategy can be quickly chosen for a new user, and an approximate frequency and current of electrical stimulation can be determined. For example, the frequency and current value thereof can be initially set to 50 Hz and 3 mA respectively to enable the new user to start effective treatment quickly and reduce exploration time.

A reinforcement learning unit, configured to optimize a long-term efficacy indicator through a reward function according to operational detection data collected by the wrist stimulator each time and a corresponding stimulation parameter set and configure a personalized stimulation strategy. The wrist stimulator continuously collects tremor data, physiological data, and other operational detection data when working each time, and correlates the data with corresponding stimulation parameter sets. The reinforcement learning unit evaluates the therapeutic effect according to these data through a reward function. When a tremor amplitude of the user significantly decreases after receiving a particular treatment, the reward function assigns a higher reward value; and conversely, the reward value decreases. For example, when the tremor amplitude of the user decreases from 10 mm to 5 mm, the reward value increases. By continually adjusting stimulation parameter set parameters, such as increasing the frequency, optimizing long-term efficacy indicators through multiple treatments, and gradually configuring a personalized stimulation strategy for the user, the present disclosure ultimately achieves precise treatment and improves the rehabilitation efficacy.

During the operation of the intelligent electrical stimulation system, the AI model module always plays a critical role. When the user receives treatment with the wrist stimulator, a built-in sensor collects tremor data at high frequency and transmits the data to the AI model module in real time. For example, 100 sets of tremor data can be collected per second, including information of displacement and acceleration. The AI model module employs a specialized algorithm to calculate the tremor amplitude in real time according to the data. For example, when the user holding a cup of water experiences tremors, a current tremor amplitude can be accurately determined by analyzing and processing the collected data.

In some examples, a waveform of the electrical stimulation pulse is one or a combination of more of a biphasic square wave, a sine wave, a pulse wave, a triangular wave, and a sharp wave, with a frequency range of 1-1000 Hz, and a current intensity of 0.1-20 mA. That is, the electric stimulation parameter set can be obtained by combining various waveforms.

In some examples, the stimulation module includes a plurality of electrode wristbands tailored to different wrist sizes, and each of the electrode wristbands is provided with one or more electrode sets for applying electrical stimulation to the target nerve; and the electrode set includes one or more electrode pads, and one electrode pad corresponds to one target nerve.

In the design of the stimulation module of the intelligent electrical stimulation system, differences in wrist sizes of different users are fully considered. A plurality of the electrode wristbands are meticulously designed to adapt to various common wrist sizes respectively. For example, a small electrode wristband is suitable for the user with a wrist circumference of 13-15 cm, a medium wristband is suitable for the user with a wrist circumference of 15-17 cm, and a large wristband is suitable for the user with a wrist circumference of 17-19 cm.

Each electrode wristband is provided with one or more electrode pads for applying electrical stimulation to the target nerves. For example, positions of the electrode pads on the medium electrode wristband are precisely designed to ensure they correspond with locations of the target nerves associated with hand tremors, such as the median and ulnar nerves. A user with a wrist circumference of 16 cm can wear the medium-sized wristband comfortably around his/her wrist, and the electrode pads are tightly fitted to skin of the user. When the wrist stimulator is activated, the stimulation module outputs electrical stimulation pulses to the target nerves through the electrode pads according to the stimulation parameter set output by the AI model module. Different sizes of electrode wristbands and rationally distributed electrode pads provide personalized and precise electrical stimulation therapies for users with different wrist sizes, thereby effectively enhancing the therapeutic effect and user experience.

Further, in some examples, a remote interconnection and collaboration system is included, the user's treatment data can be uploaded to the cloud server in real time, and a physician can monitor user progress remotely through this system and promptly intervene and adjust the therapy in case of any abnormalities, thereby ensuring the therapeutic effect for the user.

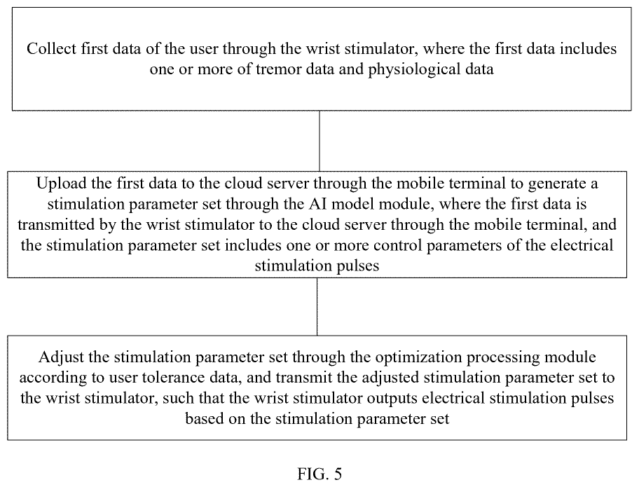

With reference to FIG. 5, the present disclosure further provides a cloud-based AI intelligent electrical stimulation control method, including:
collect first data of the user through the wrist stimulator, where the first data includes one or more of tremor data and physiological data;
upload the first data to the cloud server through the mobile terminal to generate a stimulation parameter set through the AI model module, where the first data is transmitted by the wrist stimulator to the cloud server through the mobile terminal, and the stimulation parameter set includes one or more control parameters of the electrical stimulation pulses; and
adjust the stimulation parameter set through the optimization processing module according to user tolerance data, and transmit the adjusted stimulation parameter set to the wrist stimulator, such that the wrist stimulator outputs electrical stimulation pulses based on the stimulation parameter set.

In some examples, the mobile terminal further generates pre-stored user data and/or user tolerance data based on consultation results and transmits the data to the cloud server.

After receiving the stimulation parameter set sent by the cloud server, the mobile terminal converts the stimulation parameter set into a waveform graph for display, and marks a corresponding user tolerance benchmark identifier and an original optimal stimulation identifier.

In some examples, the mobile terminal is also configured to receive the user's daily tolerance data and transmit the data to the cloud server, where the optimization processing module further adjusts the stimulation parameter set based on the daily tolerance data.

In some examples, a wearable accessory is further included, and the wearable accessory is provided with a second acquisition module in communication connection with the mobile terminal or the wrist stimulator for detecting second data of the user; and the second data includes one or more of tremor data and physiological data;
when the wearable accessory is in communication connection with the mobile terminal, the mobile terminal, upon receiving the second data, determines the user's physical condition according to the second data; and
when the wearable accessory is in communication connection with the wrist stimulator, the wrist stimulator transmits both the second data received and the first data collected to the cloud server through the mobile terminal, where the AI model module outputs a stimulation parameter set according to the first data, the second data, and the pre-stored user data.

It is understood that those of ordinary skill in art may make equivalent substitutions or modifications based on the technical solution and inventive concept of the present disclosure, and all such modifications or substitutions should fall within the scope of protection of the claims of the present disclosure.

What is claimed is:

1. A cloud-based AI intelligent electrical stimulation system, comprising:
a wrist stimulator, comprising a first acquisition module and a stimulation module, wherein the first acquisition module is configured to collect first data, and the stimulation module is configured to apply electrical stimulation pulses to one or more target nerves; the first data comprises one or more of tremor data and physiological data;
a mobile terminal in communication connection with the wrist stimulator;
a cloud server in communication connection with the mobile terminal, comprising an AI model module and an optimization processing module;
the AI model module outputs a stimulation parameter set based on the first data and pre-stored user data, and the first data is transmitted by the wrist stimulator to the cloud server through the mobile terminal; the stimulation parameter set includes one or more control parameters of the electrical stimulation pulses; and
the optimization processing module adjusts one or more control parameters of the stimulation parameter set according to user tolerance data and transmits the adjusted stimulation parameter set to the wrist stimulator, such that the wrist stimulator outputs electrical stimulation pulses based on the stimulation parameter set.

2. The cloud-based AI intelligent electrical stimulation system according to claim 1, wherein the mobile terminal further generates pre-stored user data and/or user tolerance data based on consultation results and transmits the data to the cloud server.

3. The cloud-based AI intelligent electrical stimulation system according to claim 2, wherein after receiving the stimulation parameter set sent by the cloud server, the mobile terminal converts the stimulation parameter set into a waveform graph for display, and marks a corresponding user tolerance benchmark identifier and an original optimal stimulation identifier.

4. The cloud-based AI intelligent electrical stimulation system according to claim 1, wherein the mobile terminal is also configured to receive the user's daily tolerance data and transmit the data to the cloud server, wherein the optimization processing module further adjusts the stimulation parameter set based on the daily tolerance data.

5. The cloud-based AI intelligent electrical stimulation system according to claim 1, wherein a wearable accessory is further comprised and the wearable accessory is provided with a second acquisition module in communication connection with the mobile terminal or the wrist stimulator for detecting second data of the user; and the second data comprises one or more of tremor data and physiological data;
when the wearable accessory is in communication connection with the mobile terminal, the mobile terminal, upon receiving the second data, determines the user's physical condition according to the second data and transmits the second data to the cloud server, wherein the AI model module outputs a stimulation parameter set based on the first data, the second data and the pre-stored user data;
when the wearable accessory is in communication connection with the wrist stimulator, the wrist stimulator transmits both the second data received and the first data collected to the cloud server through the mobile terminal, wherein the AI model module outputs a stimulation parameter set according to the first data, the second data, and the pre-stored user data.

6. The cloud-based AI intelligent electrical stimulation system according to claim 5, wherein the wearable accessory comprises one or more of a bracelet, glasses, an earphone, a ring, a headband, a waistbelt, a patch, and jewelry.

7. The cloud-based AI intelligent electrical stimulation system according to claim 1, wherein the target nerve is one of a radial nerve, a median nerve, or an ulnar nerve.

8. The cloud-based AI intelligent electrical stimulation system according to claim 1, wherein a training method for the AI model module comprises:
collecting multimodal data to construct a training set, wherein the multimodal data comprises data of the user's age, medical history, tremor type, real-time movement status, electrical stimulation response, and electrical stimulation parameter set;
preprocessing the training set to balance label distribution; and
training an initialized neural network based on the training set to obtain the AI model module.

9. The cloud-based AI intelligent electrical stimulation system according to claim 1, wherein the AI model module comprises:
a transfer learning unit, being configured to adapt an initial stimulation strategy for a new user based on a cross-user data generalization model; and
a reinforcement learning unit, being configured to optimize a long-term efficacy indicator through a reward function according to operational detection data collected by the wrist stimulator each time and a corresponding stimulation parameter set and configure a personalized stimulation strategy.

10. The cloud-based AI intelligent electrical stimulation system according to claim 1, wherein a waveform of the electrical stimulation pulse is one or a combination of more of a biphasic square wave, a sine wave, a pulse wave, a triangular wave, and a sharp wave, with a frequency range of 1-1000 Hz, and a current intensity of 0.1-20 mA.

11. The cloud-based AI intelligent electrical stimulation system according to claim 1, wherein the first data further comprises physiological data;
the physiological data comprises one or more of skin impedance data, therapeutic response data, electromyographic signal data, neural signal data, skin temperature data, and blood oxygen saturation data.

12. The cloud-based AI intelligent electrical stimulation system according to claim 1, wherein the stimulation module comprises a plurality of electrode wristbands tailored to different wrist sizes, and each of the electrode wristbands is provided with one or more electrode sets for applying electrical stimulation to the target nerve; and the electrode set comprises one or more electrode pads, and one electrode pad corresponds to one target nerve.

13. A cloud-based AI intelligent electrical stimulation control method, comprising:
collecting first data of the user through the wrist stimulator, wherein the first data comprises one or more of tremor data and physiological data;
uploading the first data to the cloud server through the mobile terminal to generate a stimulation parameter set through the AI model module, wherein the first data is transmitted by the wrist stimulator to the cloud server through the mobile terminal, and the stimulation parameter set comprises one or more control parameters of the electrical stimulation pulses; and
adjusting the stimulation parameter set through the optimization processing module according to user tolerance data, transmitting the adjusted stimulation parameter set to the wrist stimulator, such that the wrist stimulator outputs electrical stimulation pulses based on the stimulation parameter set.
